(19) United States
(12) Patent Application Publication
(10) Pub. No.: US 2025/0316187 A1
(43) Pub. Date: Oct. 9, 2025

(54) ALERT BUTTON TRAINING SYSTEM FOR A WEARABLE MEDICAL DEVICE AND METHOD THEREOF

(71) Applicant: WEST AFFUM HOLDINGS DAC, DUBLIN (IE)

(72) Inventors: Brian D. Webster, Mercer Island, WA (US); Jeffrey M. Boschee, Woodinville, WA (US); Pamela F. Breske, Newcastle, WA (US)

(73) Assignee: WEST AFFUM HOLDINGS DAC, DUBLIN (IE)

(21) Appl. No.: 19/098,758

(22) Filed: Apr. 2, 2025

Related U.S. Application Data
(60) Provisional application No. 63/574,477, filed on Apr. 4, 2024.

(51) Int. Cl. G09B 23/28; A61N 1/39; G09B 5/02; G09B 5/06

(52) U.S. Cl. CPC G09B 23/28; A61N 1/3904; A61N 1/3987; A61N 1/3993; G09B 5/02; G09B 5/062

(57) ABSTRACT

A medical system provides training to a patient to override therapy delivery. The medical system includes a wearable medical device, one or more remote servers, and a mobile device. The wearable device detects cardiac conditions using ECG data and delivers therapy but issues an alert prior to delivering the therapy. The wearable medical device includes an alert button that can be pressed by the patient to override the therapy delivery. The mobile device includes at least a processor configured to receive a communication to initiate a training mode from the one or more remote servers and initiate the training mode based on the communication. The mobile device further includes a user interface to provide an instruction to the patient, which instructs the patient to press the alert button when the alert is output, to override delivery of the therapy.

### CROSS-REFERENCE TO RELATED APPLICATIONS

This application claims priority to and benefit of the provisional patent application No. 63/574,477 titled “WCD SYSTEM WITH ALERT BUTTON TRAINING”, filed in the United States Patent and Trademark Office on Apr. 4, 2024. The specification of the above-referenced patent application is incorporated herein by reference in its entirety.

### TECHNICAL FIELD

The present technology relates to a wearable medical system that includes a wearable medical device in communication with a mobile device and one or more remote servers. More specifically, the wearable medical system provides alert button training to the patient to override delivery of therapy especially when false positive cardiac conditions are detected.

### BACKGROUND

Certain heart arrhythmias can disrupt blood flow, potentially leading to severe complications such as Sudden Cardiac Arrest (SCA). SCA is a life-threatening event that can cause death within minutes if not promptly treated. Therefore, patients at higher risk of SCA, such as those with a history of heart attack or a previous SCA episode, are often recommended a wearable defibrillator. Wearable defibrillator continuously monitors the patient's heart rhythm and delivers an electric shock if dangerous arrhythmias are detected. Unlike implantable devices, wearable defibrillators are non-invasive and do not require surgery, making them a viable option for patients who need temporary protection while their long-term treatment plan is determined.

However, despite their life-saving potential, wearable defibrillators present certain challenges, particularly in cases of false detections. These devices rely on sensors and algorithms to assess the patient's heart rhythm, but in some instances, external factors such as muscle movement, noise interference, or non-life-threatening arrhythmias can sometimes trigger incorrect detections of cardiac events. In such cases, the device may mistakenly prepare to administer a shock, causing unnecessary distress for the patient.

Patients who experience false alarms often report high levels of anxiety and fear, particularly if they are unsure of how to respond. If the device proceeds to deliver a shock in the absence of a true life-threatening arrhythmia, the patient may experience unnecessary physical discomfort, emotional trauma, and a loss of confidence in the device. Over time, repeated false alarms and shocks may lead to non-compliance, with some patients choosing to remove or avoid wearing the device altogether. This, in turn, ultimately increases the risk of an actual SCA going untreated.

### SUMMARY

The present disclosure relates to a medical system for providing training to a patient to override delivery of a therapy. In one aspect of the present disclosure, the medical system includes the wearable medical device, one or more remote servers, and a mobile device. The mobile device is in communication with the wearable medical device and the one or more remote servers. The wearable medical device is configured to deliver therapy to the patient upon detection of a cardiac condition of the patient based on patient electrocardiogram (ECG) data and output an alert prior to providing the therapy. The wearable medical device further includes an alert button configured to override delivery of the therapy, when pressed by the patient. The mobile device includes at least one processor configured to receive a communication to initiate a training mode from the one or more remote servers. The processor initiates the training mode based on the received communication. The mobile device further includes a user interface configured to provide an instruction to the patient upon initiation of the training mode. The instruction instructs the patient to press the alert button when the alert is output, to override delivery of the therapy.

The present disclosure further relates to the one or more remote servers operable to schedule initiation of the training mode based on a scheduled training frequency, patient use of the wearable medical device, and/or when the patient ECG data indicates that a noise threshold has been exceeded.

The present disclosure also relates to receiving the communication on the mobile device in the form of a text message or notification (e.g., push notification). The training mode initiated based on received communication includes a training video. The wearable medical device is operable to send a second communication to the mobile device when the patient presses the alert button. The second communication causes a training completion indication to be provided on the user interface. The wearable medical device further includes at least one therapy electrode in communication with an energy source. The at least one therapy electrode is configured to deliver a shock to the patient based on the patient ECG data.

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 depicts components of a medical system along with the connections therebetween, according to an embodiment of the present disclosure.

FIG. 2 depicts a communication received on a mobile device of a patient, according to an embodiment of the present disclosure.

FIG. 3 depicts a second communication sent to the mobile device by a wearable medical device, according to an embodiment of the present disclosure.

FIG. 4 depicts a communication received on a patient application installed on the mobile device, according to an embodiment of the present disclosure.

FIG. 5 depicts a second communication sent to the patient application by the wearable medical device, according to an embodiment of the present disclosure.

FIG. 6 depicts a block diagram disclosing a process of initiation of training mode, according to an embodiment of the present disclosure.

FIG. 7 depicts a block diagram disclosing a process when the patient fails to press the alert button during the training mode, according to an embodiment of the present disclosure.

FIG. 8 depicts a block diagram disclosing the initiation of a call to the patient when the patient fails to press the alert button, according to an embodiment of the present disclosure.

FIG. 9 depicts example buttons provided to the patient via the mobile device to seek medical help, according to an embodiment of the present disclosure.

FIG. 10 depicts an example method for training the patient to override delivery of the therapy, according to an embodiment of the present disclosure.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures or methods associated with the medical system has not been shown or described in detail to avoid unnecessarily obscuring descriptions of the embodiments.

Unless the context indicates otherwise, throughout the specification and claims which follow, the word “comprise” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one aspect” or “an aspect” means that a particular feature, structure, or characteristic described in connection with an embodiment is included in at least one aspect. Thus, the appearances of the phrases “in one aspect” or “in an aspect” in various places throughout this specification are not necessarily all referring to the same embodiment. Furthermore, the particular features, structures, or characteristics may be combined in any suitable manner in one or more aspects.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Certain terms and phrases have been used throughout the disclosure and will have the following meanings in the context of the ongoing disclosure:

The term “wearable medical device” refers to a medical device worn by a patient, which is designed to monitor, detect, or provide therapeutic intervention for specific medical conditions. In the context of the present disclosure, the wearable medical device is configured to detect cardiac conditions of the patient based on patient data. The wearable medical device may be worn by a patient in form of a vest or garment. Further, the wearable medical device may provide necessary therapy to the patient upon detecting the cardiac conditions. The therapy may include but is not limited to providing electrical stimulation or pacing in the form of a shock. The wearable medical device works in conjunction with a mobile device to ensure continuous patient monitoring and efficient data exchange.

The term “mobile device” refers to a portable, handheld electronic device that enables communication of a user or patient with the wearable medical device and a server. The mobile device includes hardware components such as a processor, memory, and a user interface to execute one or more functions.

The term “server” or “remote server” refers to a computer system or network server responsible for managing patient data and communications. Further, the term “remote service” refers to a service provided or performed from a distance or remotely, typically using technology such as the internet, telecommunication systems, and so on. It allows individuals or organizations to access services or support without the need to be physically present at the location where the service is being delivered. The remote service may be the service provided by the remote server.

The embodiments of the present disclosure relate to providing training to a patient using a wearable medical device. The training primarily involves teaching the patient how to use an alert button provided on the wearable medical device. Before the training is initiated, a communication is received on a mobile device (e.g., a smartphone) of the patient, where the mobile device is communicatively coupled with the wearable medical device. During the training, an instruction is provided to the patient about usage of the alert button. In other words, the patient learns how to use the alert button to abort delivery of therapy. As a result, the therapy is aborted when the patient successfully presses the alert button. In an embodiment, the communication related to initiation and completion of the training is provided via text on patient's mobile phone. In another embodiment, the communication is provided via a patient application that is paired to the wearable medical device and installed on the patient's mobile device. In yet another embodiment, the communication may also be shared with a caregiver or any other person related to the patient, especially in case the patient is not able to receive the training. In some embodiments, the communication related to the training may be provided to the patient, caregiver, or any other person related to the patient through other triggers. The other triggers may include but are not limited to scheduled reminders (e.g., timed notifications), push notifications (e.g., mobile app push notifications to remind the patient about upcoming training sessions or reminders to complete specific training tasks), device usage monitoring-based triggers (e.g., triggers based on device inactivity), training completion-based triggers, in-app alerts (e.g., pop-up messages), event-based triggers (e.g., triggers based on health status changes), personalized triggers (e.g., triggers based on patient's preferences), and/or triggers by healthcare provider or medical device administrator.

When the training is provided to the patient, he/she can press the alert button and divert the shock therapy, if not required. For instance, if the patient is feeling well, or if a false positive is generated by the wearable medical device because of some misalignment or wrong position while wearing the medical device, and so on. In such cases, the patient may press the alert button to prevent the shock therapy, as administering unnecessary shocks may cause discomfort to the patient.

In some embodiments, the patient is contacted in case the patient does not respond to the notifications for a predefined number of times. For example, if the patient is notified five times (a predefined value) about abnormal cardiac data and the need for shock therapy, or if the patient does not respond to notifications regarding the regular training sessions on how to use the wearable medical device. If the patient does not respond to any of these notifications, a call is scheduled with the patient, to understand the reason behind their lack of acknowledgment. If the patient still does not answer the call, another call is initiated with the caregiver or any other personnel related to the patient.

In some embodiments, the working of the medical system along with the initiation of training mode is monitored by the caregiver, support personnel, or a clinician, who is notified by the remote server when the patient has exceeded threshold(s). In response to the notification, the caregiver or support personnel can launch the training mode. In addition, the support personnel can also launch training mode when a patient calls for support. In some embodiments, the clinician may review episode data received by the remote server. Based on the reviewed episode data, the clinician may launch the training mode in response to determining that an inappropriate shock is delivered. In some embodiments, the support personnel and/or clinicians can also set up the remote server to send push notifications or other triggers (e.g., text messages) to the patient. The frequency of push notifications or other triggers may be set as recurring (or reoccurring) at regular intervals (e.g., daily, weekly, or monthly).

In still other embodiments, the remote service is configured to enable support personnel and/or clinicians to configure the remote server to send single or scheduled training text messages or push notifications on per patient or clinical basis. Within the context of this disclosure, the terms “text message” and “notification” are to be construed interchangeably at least due to their functional similarity. In embodiments in which the mobile device has an installed patient application, the remote server may be configured to enable support personnel and/or clinicians to configure the remote server to set up the patient application to launch one or more training sequences on per patient or clinical basis and according to a specified schedule.

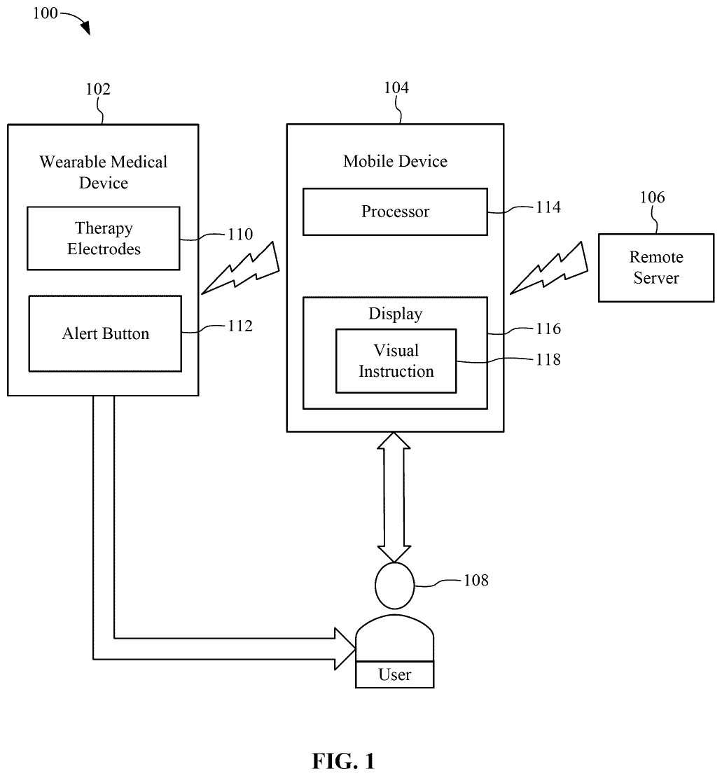

FIG. 1 depicts a medical system 100 including a wearable medical device 102, a mobile device 104, and a remote server 106 along with the connections therebetween, according to an embodiment of the present disclosure. For brevity, only one server, mobile device, and wearable medical device are depicted in FIG. 1. However, in some implementations, the medical system 100 may include multiple servers, mobile devices, and/or wearable medical devices without departing from the scope of the present disclosure.

The wearable medical device 102 may be worn by a user 108. The non-limiting examples of the user 108 may include a patient, a physician, a medical staff member, a wearer, a support staff, a caretaker, a family member, a medical professional, a healthcare provider, or the like. Additionally, the user 108 may be responsible for ensuring proper placement and maintenance of the wearable medical device 102, which may include ensuring that the wearable medical device 102 is securely affixed to the body and functioning correctly. Since the user 108 may correspond to the patient 108 using the wearable medical device 102, therefore, within the context of the disclosure, the terms “user” and “patient” may be used interchangeably.

As shown in FIG. 1, the wearable medical device 102 provides real-time cardiac monitoring and life-saving intervention for the patient 108 at risk of sudden cardiac arrest. For instance, the patient 108 may have recently suffered from a heart attack and a clinician may have asked the patient 108 to wear the wearable medical device 102, so that the patient 108 can be monitored to circumvent the risks associated with repeated heart attack episodes. This preventive measure prevents the risk of certain cardiac conditions, as the patient 108 can be given a shock therapy, if the wearable medical device 102 senses any risk of cardiac arrest. In other embodiments, the wearable medical device 102 may provide pacing therapy, or both defibrillation and pacing therapy to treat a cardiac condition of the patient 108. Depending on the context, the term “wearable medical device” may be interchangeably used as “wearable cardioverter defibrillator (WCD)” in the present disclosure.

The wearable medical device 102 includes at least one therapy electrode 110 for delivering an electric shock when the severe cardiac event is detected based on cardiac data of the patient 108 that includes an electrocardiogram (ECG) data of the patient 108. The therapy electrode 110 is positioned on the patient's body to ensure maximum conductivity and effectiveness. The therapy electrode 110 may be made from conductive material that allows efficient transmission of electrical energy while minimizing skin irritation for long-term wear. The therapy electrode 110 may be connected to an energy source (not shown in the figure) to generate and store the necessary electrical charge required for defibrillation. The energy source is typically a battery-powered capacitor system capable of delivering high-voltage shocks when needed. When the wearable medical device 102 identifies a life-threatening arrhythmia, such as ventricular fibrillation (VF) or ventricular tachycardia (VT), the wearable medical device 102 rapidly initiates the energy source and delivers the electric shock to the patient 108 via the therapy electrode 110. The therapy electrode 110 releases measured electrical pulse designed to reset the electrical activity of the heart of the patient 108 to restore a normal cardiac rhythm.

The wearable medical device 102 continuously monitors the patient's ECG data by analyzing the electrical signals of the patient's heart in real-time. The wearable medical device 102 detects irregularities that may indicate a dangerous cardiac condition. For instance, the irregularities in the ECG data can indicate various cardiac conditions that require medical attention. Such irregularities in the ECG data may include but are not limited to arrhythmias, such as atrial fibrillation (AF), where the heart beats irregularly and rapidly due to chaotic electrical signals in the atria, and ventricular tachycardia (VT), a potentially life-threatening condition where the ventricles beat too quickly, reducing blood flow. Another abnormality is bradycardia, where the heart beats too slowly, often due to issues with the sinoatrial (SA) node or conduction pathways. ST-segment elevation or depression in the ECG data can indicate myocardial infarction (heart attack), while prolonged QT intervals may suggest a risk of sudden cardiac arrest due to dangerous ventricular arrhythmias. Premature ventricular contractions (PVCs) or premature atrial contractions (PACs) are irregular extra beats that may signify underlying heart disease or electrolyte imbalances. Additionally, bundle branch blocks (BBB), where electrical impulses are delayed or blocked in the heart's conduction system, can be a sign of structural heart disease. T-wave inversions or abnormal P-wave morphology may indicate conditions such as ischemia, electrolyte disturbances, or atrial enlargement. By analyzing these abnormalities, the wearable medical device 102 can detect potentially dangerous cardiac conditions and avoid fatality by providing shock therapy to the patient 108.

In addition to the therapy electrode 110, the wearable medical device 102 includes an alert button 112. While the term “alert button” is used herein, in some embodiments, the alert button 112 may have more than one button that must be operated to perform a desired function such as aborting or diverting a shock. Accordingly, the term “alert button” is not intended to be limiting, and any switch, control, activator, input mechanism, or push mechanism performing a similar function is considered within the scope of the embodiments described herein.

If an abnormal heart rhythm is detected, the wearable medical device 102 prepares to deliver the shock therapy but first issues a pre-therapy alert to notify the patient 108. This alert is typically provided through multiple ways, including, but not limited to, audible alarms, vibrations, and visual notifications to ensure the patient 108 is aware of the impending electric shock. The alert serves as a safeguard, allowing the patient 108 to assess their condition. If the patient 108 is conscious and determines that the alert was triggered due to a false alarm, such as excessive movement, noise interference, or a non-life-threatening arrhythmia, the patient 108 can press the alert button 112 to override the electric shock therapy. Pressing the alert button 112 prevents unnecessary delivery of the shock therapy and reduces patient's discomfort and anxiety, and/or battery depletion associated with the wearable medical device 102. However, if the patient 108 is unresponsive and does not cancel the alert within a designated time frame, the wearable medical device 102 may proceed with therapy delivery automatically to prevent a potentially fatal cardiac event. There may be a situation where the ECG data recorded by the wearable medical device 102 is incorrect because of multiple factors. For instance, if the patient 108 is exercising or wearing the wearable medical device 102 incorrectly, the ECG data may produce incorrect results. During these instances, the patient 108 can make use of the alert button 112 to abort the shock therapy.

The integration of the therapy electrode 110, the energy source, continuous ECG data monitoring, and the alert button 112 makes the wearable medical device 102 reliable for managing life-threatening cardiac conditions. The therapy electrode 110 ensures effective electric shock delivery, while the alert button 112 provides patient-controlled intervention, balancing safety and comfort.

The medical system 100 further includes the remote server 106 to manage and optimize the functionality of the wearable medical device 102 by enabling real-time data processing, communication, and patient training. The remote server 106 is responsible for storing, analyzing, and transmitting important patient data collected from the wearable medical device 102. The medical system 100 may include one or more remote servers 106 to handle large volumes of the patient's ECG data, therapy logs, and performance metrics of the wearable medical device 102. The remote server 106 act as a moderator, ensuring seamless communication between the wearable medical device 102, the patient's mobile device 104, and healthcare providers. By continuously monitoring and analyzing data, the remote server 106 may contribute to both proactive patient care and enhanced device management.

Additionally, the remote server 106 facilitates real-time data transmission and feedback, allowing for timely updates and instructions to the patient 108. In an embodiment, the remote server 106 may be implemented using a remote patient data platform such as Kestra CareStation® which is developed and offered by Kestra Medical Technologies, Inc. of Kirkland, Washington (hereinafter referred to as “Kestra”). In some embodiments, the remote server 106 may be implemented using a web service such as Amazon Web Services®, Microsoft Azure®, Google Cloud Platform, or any other cloud platform provider.

In some examples, the remote server 106 may be implemented as a cloud or an on-demand system that is operated by an organization or a third-party on behalf of the organization. In some examples, the remote server 106 may be implemented in a cloud environment. For simplicity, the remote server 106 depicted in FIG. 1 may be a cloud environment that is intended to represent various forms of servers including a web server, an application server, a proxy server, a network server, a server pool, and/or the like.

In some embodiments, the remote server 106 may be configured to collect the patient data including but not limited to patient vitals, ECG data associated with arrhythmias detected by the wearable medical device 102, and location information of the wearable medical device 102 or the mobile device 104. Further, in some embodiments, the remote server 106 makes the aforementioned collected data accessible to a physician of the patient 108 and members of a clinical team that are remotely located. The collected patient data is then utilized for detection, prognosis, and prediction of any present or upcoming cardiac conditions.

Further, for the sake of brevity, the remote server 106 is indicated as a single remote server 106 in FIG. 1, however there may be multiple remote servers, either at the same geographical location or at different geographical locations operating and coordinating in a distributed manner to achieve server functionality, without limitation. Depending on the context, “remote server” and “one or more remote servers” are being used in the present disclosure.

The medical system 100 further includes the mobile device 104 for establishing communication between the wearable medical device 102, the remote server 106, and the patient 108. The mobile device 104 may include a smartphone, a laptop, a desktop, an iPad, a tablet, or any other communication device that can be operatively coupled to the wearable medical device 102. In an embodiment, the mobile device 104 is provided by Kestra, which may not have a phone number or Subscriber Identity Module (SIM) card for sending or receiving Short Message Service (SMS) messages. Since the application-based system relies on internet connectivity rather than cellular text services, the patient 108 can still receive training messages, ensuring that the patient 108 is not excluded from critical training opportunities. This inclusive approach helps broaden access to essential safety training for all patients, regardless of their personal device limitations.

The mobile device 104 provides training notifications and instructional guidance to the patient 108 and ensures that the patient 108 is adequately informed about how to operate the wearable medical device 102 properly. The mobile device 104 includes at least a processor 114 and a user interface (e.g., shown as display 116), where the processor 114 is configured to handle communications, running a patient application, processing the patient data captured from the wearable medical device 102, and executing commands from the remote server 106. As an example, the patient application may be ASSURE® Patient Application provided by Kestra but may include any other application designed to perform similar functionality. The display 116, on the other hand, serves as a primary user interface through which the patient 108 interacts with the medical system 100, and receives alerts, messages, and training instructions. Although display 116 is shown as an example of the user interface of the mobile device 104, within the context of the present disclosure, the user interface may be configured to provide a visual output (e.g., a display), an audio output (e.g., a speaker), and/or a haptic output (e.g., vibration), without limitation.

The mobile device 104 may be continuously in communication with the wearable medical device 102 and/or the remote server 106, allowing for real-time data exchange and prompt response to any alerts or instructions. This connectivity ensures that the patient 108 remains informed and can take immediate action if necessary. When the remote server 106 determines that training is needed, whether due to a scheduled training session, patient inactivity, or an ECG noise threshold being exceeded, the remote server 106 sends a communication to the mobile device 104, instructing initiation of a training mode. The mobile device 104 then receives this communication and triggers the training mode accordingly.

Although the training mode is initiated on the mobile device 104 based on the communication received from the remote server 106, it should be noted that, in some embodiments, the training mode may be automatically initiated by the medical system 100 based on one or more predefined criteria. One of the exemplary scenarios when the training mode is initiated automatically may include but is not limited to, the patient 108 has exceeded certain predetermined threshold(s) for alerts, noise, and so on. For instance, if the wearable medical device (or WCD) 102 detects excessive environmental noise or signal interference (e.g., radio frequency interference, loud background sounds that might mask alerts, and so on), the medical system 100 assumes the patient 108 is in an environment where alerts are hard to perceive. As a result, the medical system 100 initiates training mode to educate the patient 108 on how to move to a quieter environment.

In some embodiments, the patient application installed on the mobile device 104 is configured to detect the alert, compare the alert to a predetermined threshold, and in response to the comparison, launch the training mode. The patient application may be designed to continuously monitor alerts generated by the wearable medical device 102. These alerts are assessed against the predetermined thresholds, which may include factors such as the frequency of alerts, severity of detected cardiac event(s), device status notifications, and the like. In an instance, the training can also be provided after a cardiac episode has occurred if the patient 108 did not divert the therapy (either after a shock therapy is complete, or after one or more on spontaneous conversion episodes).

Once the training mode is initiated, the mobile device 104 provides an instruction (e.g., a visual instruction 118) on the display 116 to guide the patient 108 through the necessary steps. Although the embodiments are described with respect to the instruction being depicted as visual instruction 118 displayed on the display 116, the instruction may be provided as an audio instruction, haptic-based instruction, and/or any other form of instruction, without limitation. The primary purpose of this training is to ensure that the patient 108 knows how to respond when the alert button 112 on the wearable medical device 102 is triggered. Specifically, the training mode presents the visual instruction 118 on the display 116, directing the patient 108 to press the alert button 112 when an alert is output. Pressing the alert button 112 at the right time allows the patient 108 to override the delivery of the shock therapy in cases where the electric shock may be unnecessary, such as when a false alarm occurs due to noise interference in the ECG data. In other words, the false alarm occurs when the ECG exceeds a threshold, where the threshold may include one or more of: a threshold number of noise events, a threshold number of alerts, a threshold activity level (as detected by an accelerometer embedded within the wearable medical device 102).

The medical system 100 described in FIG. 1 is configured to provide training to the patient 108 to educate the patient 108 about the usage of the alert button 112. During training, the patient 108 receives the visual instruction 118 on the display 116 of the mobile device 104. The training may include comprehensive instructions on the location of alert button 112 and when the patient 108 should press the alert button 112. The training may also educate the patient 108 on recognizing the symptoms that indicate when the therapy is needed and the conditions under which the therapy should be aborted by pressing the alert button 112. In addition, the training ensures that the patient 108 is comfortable and knowledgeable about operating the wearable medical device 102 during daily activities. Therefore, the training mode allows the patient 108 to use the alert button 112 and abort the anticipated delivery of the shock therapy with false alarms. As a result, the wearable medical device 102 delivers therapy to the patient 108 only when needed, which avoids the occurrence of shock delivery when the patient's cardiac condition is normal. In other embodiments, the training may be provided to the patient 108 on garment fitting, assembly of components into the garment, connection of therapy cable to a monitor (e.g., a plug is not fully inserted), and the like.

The communication from the remote server 106 can take different forms, ensuring that the patient 108 receives the message through multiple channels. In an embodiment, a method of communication is a text message displayed on the display 116 of the mobile device 104, which provides clear, written instructions. In another embodiment, the communication may be in the form of the notification delivered through the patient application on the mobile device 104. This allows for more interactive engagement and ensures that the patient 108 receives the message even if text messaging is unavailable. In other embodiments, family members and/or caregivers are provided with a “Caregiver Application” to provide functionality to family members or the patient 108. In such instance, the caregiver can initiate the training mode for the patient 108 when the ECG data crosses a threshold. In such instance, the medical system 100 is configurable to setup a similar training sequences and/or reminders for patients' family members and/or caregivers to train them via text message or other suitable modes of communication including but not limited to videos, animations, audio messages, and the like.

To enhance patient's comprehension and engagement, the training mode may also include a training video as part of the visual instruction 118. This video demonstrates the proper steps to follow, including when and how to press the alert button 112, supporting the importance of the action. By combining text-based instructions, application notifications, and multimedia content, the mobile device 104 ensures that the patient 108 is effectively trained. In some embodiments, the training mode may include a training bot (such as a chatbot) configured to interact with the patient 108 to educate him/her on the usage of the alert button 112.

The mobile device 104 functions as a training and communication tool that bridges the gap between the patient 108, the wearable medical device 102, and the remote server 106. By receiving communications from the remote server 106, initiating training sequences, and delivering clear visual instruction(s) 118, the mobile device 104 enhances patient's safety, reinforces proper usage of the wearable medical device 102, and ultimately helps prevent unnecessary electric shocks, thus improving the overall effectiveness of the wearable medical device 102. In some embodiments, the mobile device 104 may be a dedicated trainer device. In an example, the trainer device may trigger training sequences through audio prompts. In another example, the trainer device may use cellular radio service to synchronize data with a remote platform (e.g., server) or application, and/or to receive training-related or patient-related updates.

The wearable medical device 102 not only detects cardiac abnormalities and delivers the shock therapy but also communicates with the mobile device 104 to ensure that the patient 108 properly interacts with the medical system 100. For example, when the patient 108 presses the alert button 112, the wearable medical device 102 detects this action and immediately transmits a second communication to the mobile device 104. This communication serves as an acknowledgment that the patient 108 has correctly followed the instructions and taken the appropriate action. The transmission of the second communication ensures that the medical system 100 recognizes and records the patient's response.

Upon receiving the second communication, the mobile device 104 processes the patient's data and triggers a training completion indication on the display 116. This indication serves multiple purposes such as but not limited to providing positive reinforcement to the patient 108, confirming that the medical system 100 has recognized patient's action, and reassuring the patient 108 that he/she has correctly followed the training steps. The training completion indication may be shown to the patient 108 as a pop-up message, a notification, an audio cue like a guitar strum sound, and so on. Such a feedback mechanism helps reinforce patient's learning and ensures that the patient 108 gets familiarized about the procedure for overriding therapy if necessary. Further, once the training is completed by the patient 108, the training completion data (e.g., completion status, timestamps, success rates, etc.) may be transmitted from the mobile device 104 to the remote server 106. The training completion data may also include specific metrics such as how long the patient 108 took to complete the training, whether they answered all the questions correctly, or whether they engaged with the content. The remote server 106, upon receipt of the training completion data, may process the training completion data to assess whether the patient 108 has complied with the training requirements or guidelines. The compliance process may include assessing whether all necessary training steps were completed correctly and in entirety, comparing training outcomes against predefined thresholds or standards set by medical professionals or device manufacturers, and providing compliance status. In an embodiment, if the patient 108 has complied with the training requirements, the remote server 106 may update the patient's training record and send a notification to the mobile device 104, which is displayed to the patient 108 as the training completion indication on the display 116. In another embodiment, if the patient 108 has not complied with the training requirements or if the training was incomplete, additional training sessions or reminders could be triggered to ensure that the patient 108 is properly trained.

By incorporating this feedback loop, the medical system 100 enhances patient confidence, compliance, and engagement. This helps ensure that patient 108 does not just receive training instructions but also practices and confirms their understanding of how to use the alert button 112 effectively. Additionally, the ability of the wearable medical device 102 to send a confirmation communication allows healthcare providers and monitoring systems to assess whether additional training is needed, ensuring that the patient 108 is well-prepared for real-life emergency scenarios. This feature ultimately contributes to patient safety, better device usage, and reduced risk of unnecessary therapy delivery.

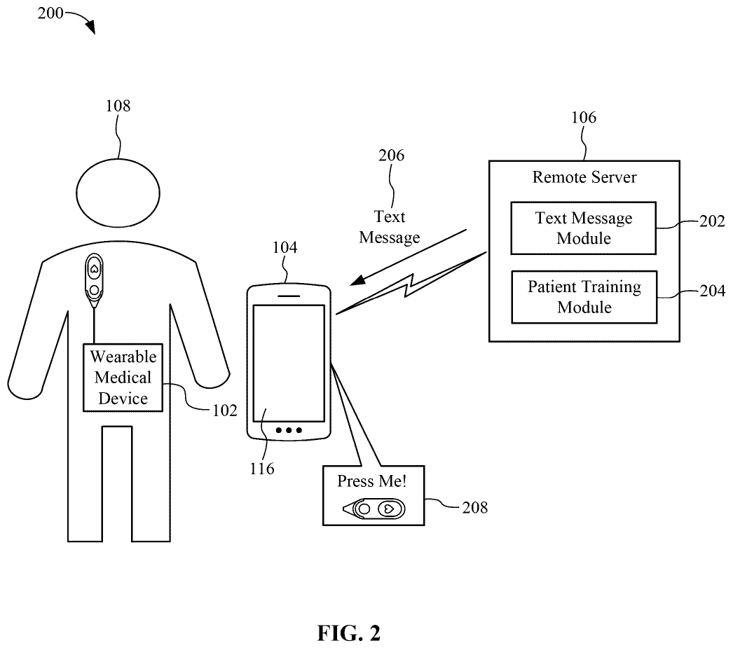

FIG. 2 depicts an exemplary embodiment where communication is received on the mobile device 104 of the patient 108 to initiate the training mode, according to an embodiment of the present disclosure. FIG. 2 is explained in conjunction with FIG. 1. Based on the received communication, the mobile device 104 initiates the training mode on the mobile device 104. More specifically, the remote server 106 sends the communication to the mobile device 104 to initiate the training mode, based on received ECG data of the patient 108 or a predefined training schedule.

In one embodiment, the wearable medical device 102 is connected to the mobile device 104 via Bluetooth®. However, those skilled in the art will appreciate that any suitable communication method or technique may be utilized to operably couple the wearable medical device 102 to the mobile device 104.

In an exemplary scenario 200, as depicted in FIG. 2, training is initiated on the mobile device 104 of the patient 108 using the wearable medical device 102 through text-based communication. For example, the wearable medical device 102 may be WCD 102, such as but not limited to ASSURE® WCD provided by Kestra. The medical system 100 integrates the wearable medical device 102 (i.e., WCD), the mobile device 104 (e.g., a smartphone), and the remote server 106 (e.g., Kestra CareStation® or Salesforce CRM). The remote server 106 is equipped with specialized modules for sending text-based communication (may be referred to as ‘text message’) to the mobile device 104. In some embodiments, and as a matter of convention used herein, instances of a “module,” “specialized modules” may be referred to as ‘software,’ ‘program,’ or other similar terms. Generally, the module includes a set of the instructions so as to offer or fulfill a particular functionality. Embodiments of modules and the functionality delivered are not limited by the embodiments described in the present disclosure. Typically, the set of instructions are stored in a memory and can be executed by one or more processors, where the processor and memory may be the components of the remote server 106 or may be operably coupled to the remote server 106. Further, ‘a set of such instructions’ can also be called a program. The instructions, which may also be referred to as “software,” generally provide functionality by performing acts, operations and/or methods as may be disclosed herein or understood.

As shown in FIG. 2, the remote server 106 includes a text message module 202 and a patient training module 204. The text message module 202 is configured to generate one or more text messages 206, which are displayed to the patient 108 via the display 116 of the mobile device 104. For the sake of brevity, a text message 206 is displayed in FIG. 2 as being triggered by the text message module 202 of the remote server 106. Based on the communication in form of the text message 206 received from the remote server 106, the training mode is initiated on the mobile device 104 and a visual instruction 208 is displayed to the patient 108 via the display 116. As shown herein, the visual instruction 208 includes a pictorial representation of the alert button 112 along with a text message ‘Press Me!’ displayed therein. Further, the patient training module 204 is configured to train the patient 108 on how to use the alert button 112 placed within the wearable medical device 102. In the present example, the text message 206 is provided to the patient 108 as a part of training to use the alert button 112. Without limitation, the communication is not limited to the text message 206 and other types of communication messages may include audio, video message, Multimedia Messaging Service (MMS), links to various types of files (WAV, MP3, etc.), and so on.

In practice, when the patient 108 wears the wearable medical device 102, the wearable medical device 102 continuously monitors patient's cardiac activity for any signs of arrhythmias or other cardiac events. If a potential cardiac event is detected, the wearable medical device 102 initiates a shock sequence to deliver shock therapy. However, the wearable medical device 102 includes the alert button 112 that allows the patient 108 to override the therapy if the patient 108 believes the alert is a false alarm. To ensure that the patient 108 is well-prepared to use the wearable medical device 102, the remote server 106 sends the text message 206 to the patient's mobile device 104 to initiate the training mode.

For example, as shown here, the training mode provides a visual instruction 208 in the form of a simple SMS containing an emoji or graphic representation of the alert button 112 accompanied by the text “Press Me!” to reinforce the action that the patient 108 should take when an alert is generated. Such short message visually prompts the patient 108 to press the alert button 112 in case of false alarms.

In other embodiments, the visual instruction 208 might be split into multiple texts to provide more detailed instructions or reminders. For example, the visual instruction 208 might include a link to an audio file that plays the sound of the shock alert. For instance, the visual instruction 208 may include a text message “Listen to this alert sound and press the Alert Button if you hear it.” By clicking on the link provided in the text message, the patient 108 can listen to the alert tone, helping the patient 108 to become familiar with the sound he/she needs to respond to during a real emergency. Such an alert tone ensures that the patient 108 recognizes the alert instantly, reducing hesitation or confusion when an actual cardiac event occurs. Alternatively, instead of the link, the remote server 106 might send an MMS message that directly includes the audio file, such as a WAV or MP3, eliminating the need for the patient 108 to navigate external links.

Additionally, the remote server 106 can enhance training by sending MMS messages containing animations or videos. For example, an MMS could feature a short animation showing the alert button 112 being pressed, causing the alert to immediately stop. This type of visual and dynamic content helps the patient 108 associate the alert button press with silencing the alert and stopping the shock sequence, reinforcing the correct action through a clear and memorable demonstration. Such a multimedia message provides engaging learning, thus making the training more effective.

Through the abovementioned varied communication methods, namely, text messages, audio files, animations, and so on, the patient 108 receives easily understandable training. This not only helps the patient 108 feel more confident in managing the wearable medical device 102 but also minimizes the risk of inappropriate therapy delivery due to patient's unawareness. By familiarizing the patient 108 with the alerts and the proper response in a non-stressful setting, the medical system 100 significantly enhances both safety and efficacy in managing cardiac emergencies.

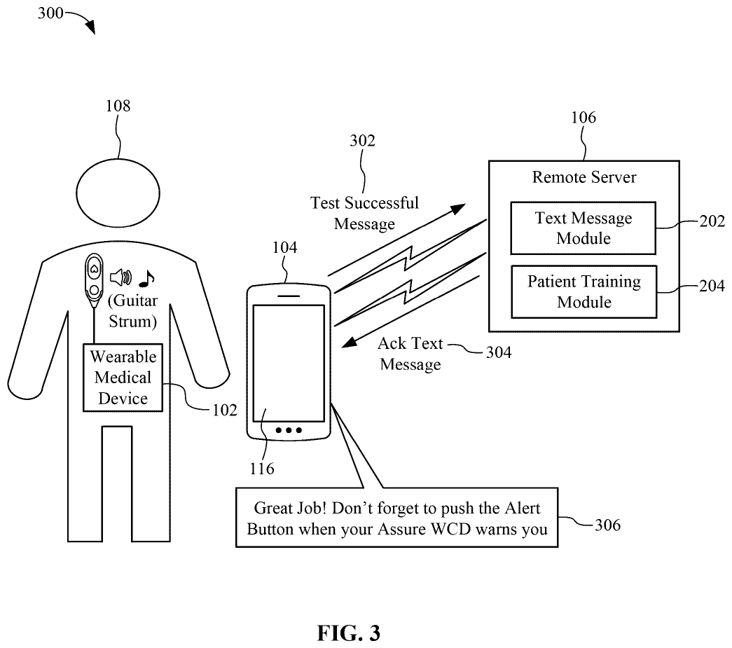

FIG. 3 depicts how the wearable medical device 102 responds when the patient 108 correctly presses the alert button 112, according to an embodiment of the present disclosure. FIG. 3 is explained in conjunction with previous figures.

In FIG. 3, an example scenario 300 is demonstrated for providing real-time feedback and reinforcement to the patient 108 using the wearable medical device 102. When the patient 108 correctly presses the alert button 112 during training, the wearable medical device 102 sends a second communication to the paired mobile device 104, which then sends a confirmation message 302 (e.g., “Test Successful” message) to the remote server 106, such as the Kestra CareStation® remote data platform. Upon receiving the confirmation message 302, the patient training module 204 triggers an acknowledgment text message 304 to the patient's mobile device 104. For example, the patient 108 might receive a message 306 saying “Great Job! Don't forget to push the Alert Button whenever your ASSURE WCD warns you.” Such positive reinforcement helps the patient 108 feel confident to be able to respond correctly during an actual cardiac event. It should be noted that the acknowledgment text message 304 received on the mobile device 104 may use a different language and format based on patient's preferences or language settings.

In an embodiment, the acknowledgment text message 304 could be sent as an MMS instead of a simple SMS. The MMS might include an audio clip of the distinctive “guitar strum” sound that the wearable medical device 102 plays, confirming proper functioning of the medical system 100. The audio clip serves as an additional layer of positive feedback, helping the patient 108 associate the pressing of the alert button 112 with the reassurance that the medical system 100 is operating as intended. For instance, hearing the familiar guitar strum reinforces the idea that the patient 108 has taken correct action.

In some embodiments, when the remote server 106 sends training instructions to the mobile device 104, the mobile device 104 triggers the wearable medical device 102 to play guitar strum sound immediately after the patient 108 presses the alert button 112. Such synchronization between the mobile device 104 and the wearable medical device 102 ensures that patient 108 receives instant feedback, making the training experience more intuitive and encouraging.

In another embodiment, the mobile device 104 can independently recognize when the training is initiated. For example, if the remote server 106 sends both a “Press Me!” text message and a notification to the mobile device 104, the mobile device 104 detects that the patient 108 is undergoing training. When the patient 108 successfully presses the alert button 112, the wearable medical device 102 triggers the mobile device 104 to play guitar strum sound. This approach not only confirms that the alert button 112 was pressed correctly but also helps the patient 108 become familiar with the alert sound associated with proper wearable medical device 102 operations.

If the patient 108 fails to press the alert button 112 within a predefined time limit after receiving a training message, the medical system 100 is designed to provide additional prompts or alerts. In such cases, the medical system 100 automatically sends a follow-up text message reminding the patient 108 to press the alert button 112. The follow-up message might use different wordings to capture the patient's attention. Exemplary follow-up message may include text such as, “Reminder: Please press the alert button as part of your training. This is your second reminder.” Such variation in language helps prevent the messages from being perceived as repetitive or easy to ignore.

In situations where the patient 108 does not respond to multiple training-related text messages, the medical system 100 is equipped to escalate the instance to a human representative. For example, if the patient 108 ignores multiple instructions, a customer support representative may contact the patient 108 on the mobile device 104 via a call to understand the underlying issue. During this call, the customer support representative might provide verbal guidance, answer questions, and ensure the patient 108 understands how and when to use the alert button 112. This personalized approach helps address any misunderstandings or technical difficulties the patient 108 might be experiencing, ensuring that the patient 108 is fully prepared to use the wearable medical device 102 effectively in a real emergency.

Through these layered and responsive training methods, including text messages, audio clips, and potential human intervention, the medical system 100 ensures that the patient 108 is well-trained and confident in managing the wearable medical device 102, particularly in critical situations. Such medical system 100 enhances patient's safety by increasing the likelihood of timely and correct responses by the patient 108 during critical cardiac events.

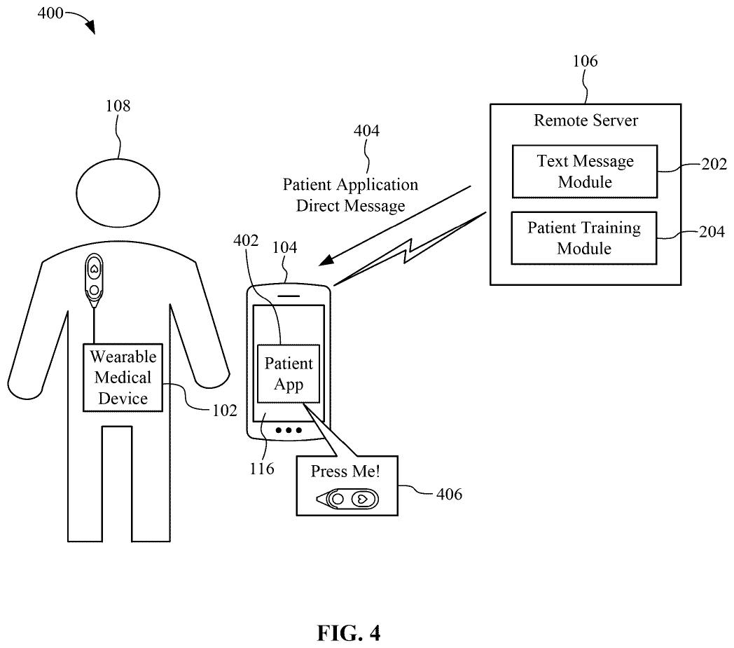

FIG. 4 depicts an exemplary scenario 400 where a patient application 402 (shown as ‘Patient app’ 402) is used to receive communication and visual instruction during the training mode, according to an embodiment of the present disclosure. FIG. 4 is explained in conjunction with previous figures.

In this exemplary scenario 400, the medical system 100 enhances patient training by sending a direct message 404 to the patient 108 via the patient application 402, instead of traditional SMS or text message 206 sent on the mobile display 116 (as shown in FIG. 2). The core functionality remains similar to previous examples, where the patient 108 receives visual instruction 118 (shown as 208 in FIG. 2) to press the alert button 112 during the training mode. However, by utilizing the patient application 402 for the direct message 404, the medical system 100 gains additional flexibility. For instance, rather than a simple SMS with text and emojis, the direct message 404 can include more engaging content. Such approach allows for richer, more visually appealing communication, which can improve patient's engagement and comprehension.

In another embodiment, the direct message 404 received on the mobile device 104 initiates the training mode where a visual instruction 406 in the form of GIF instead of a static emoji is displayed to the patient 108. For example, the GIF 406 could show a realistic image of the alert button 112, accompanied by an animation of a finger pressing it. This visual demonstration helps the patient 108 understand exactly what they need to do, making the training more intuitive and memorable. Such enhancements are particularly valuable for the patient 108 who might struggle with text-only instructions or for those who learn better through visual clues. By providing a clear and dynamic illustration of the button-pressing action, the medical system 100 helps the patient 108 feel more confident to respond correctly during a real cardiac event.

One significant advantage of using the patient application 402 direct messaging capabilities is that it accommodates patient 108, who may not own a smartphone with text messaging capabilities. For instance, the patient 108 may use a mobile device provided by Kestra or service provider, which may not have a phone number or SIM card for sending or receiving SMS/text messages. Since the app-based medical system 100 relies on internet connectivity rather than cellular text services, the patient 108 can still receive training communication and instructions, ensuring that the patient 108 is not excluded from critical training opportunities. Such inclusive approach helps broaden access to essential safety training for all patients, regardless of their personal device limitations.

Further enhancing the system's accessibility, the patient application 402 can be configured to play audio messages that read the training instructions aloud. For example, when the patient 108 receives the direct message 404, the patient application 402 initiates the training mode and automatically plays an audio including training instructions such as, “Please press the Alert Button when you hear the alert sound.” Such audio instruction is particularly beneficial for the patient 108 who has visual impairments or finds it easier to follow spoken instructions. Additionally, some embodiments include short audio clips within the direct message 404 itself, providing more context and realism to the training. For instance, the direct message 404 may say, “When you hear this from your ASSURE system,” followed by an actual audio clip of the shock alert announcement and then continue with, “You must immediately press the Alert Button. Practice pressing the Alert Button now.” Such training instructions simulate real-life scenarios, helping the patient 108 build a stronger association between the alert sound and the required action.

In some cases, if the patient 108 does not press the alert button 112 during an actual cardiac event, a shock is delivered and the patient application 402 displays the training content. One exemplary training content may include a button simulation exercise or a short training video reminding the patient 108 of the correct steps to take when they hear the alert. For example, the patient application 402 could display a simulation of the alert button 112 on the display 116, prompting the patient 108 to practice pressing the alert button 112 in response to a simulated alert sound.

By integrating the direct message 404, audio instructions, and post-event training into the patient application 402, the medical system 100 provides a user-friendly training experience. The flexibility of the app-based approach ensures that all the patients, regardless of their smartphone capabilities, can receive and respond to training instructions effectively. This combination of visual, auditory, and interactive training methods enhances the likelihood that the patient 108 will respond promptly and correctly during actual cardiac emergencies, ultimately improving their safety and outcomes.

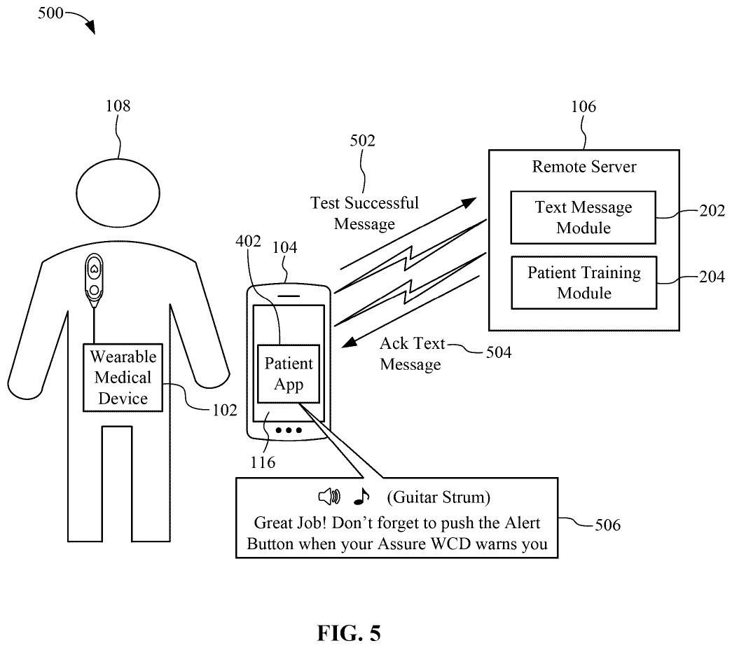

FIG. 5 depicts an exemplary scenario 500 on how the wearable medical device 102 responds when the patient 108 correctly presses the alert button 112, according to an embodiment of the present disclosure. FIG. 5 is explained in conjunction with previous figures.

The exemplary scenario 500, in FIG. 5, demonstrates a communication flow of providing real-time feedback and reinforcement to the patient 108 using the wearable medical device 102. When the patient 108 correctly presses the alert button 112 during training, the wearable medical device 102 sends a second communication to the paired mobile device 104 confirming this action, and the mobile device 104 sends a confirmation message 502 (e.g., “Test Successful Message”) to the remote server 106, such as the Kestra CareStation® remote data platform, using a text. Upon receiving this confirmation, the patient training module 204 of the remote server 106 triggers an acknowledgment text message 504 to the patient's mobile device 104. For example, the patient 108 might receive a message 506 saying, “Great Job! Don't forget to push the Alert Button whenever your ASSURE WCD warns you.” This positive reinforcement helps the patient 108 feel confident in their ability to respond correctly during an actual cardiac emergency.

In an embodiment, the acknowledgment text message 504 could be sent as an MMS instead of a simple SMS. The MMS may include an audio clip of the distinctive “guitar strum” sound that the wearable medical device 102 plays when the wearable medical device 102 verifies correct functionality. This audio cue serves as an additional layer of positive feedback, helping the patient 108 associate the alert button 112 press with the reassurance that the wearable medical device 102 or medical system 100 is operating as intended. For instance, hearing the familiar guitar strum reinforces the idea that the patient 108 has taken the correct action.

In another embodiment, the wearable medical device 102 is allowed to detect when the training mode is initiated on the mobile device 104. For example, when the remote server 106 communicates the mobile device 104 to initiate the training mode via the patient application 402, the wearable medical device 102 invokes the patient application 402 play the guitar strum sound as soon the alert button 112 is pressed correctly. This synchronization between the patient application 402 and the wearable medical device 102 (e.g., WCD) ensures that patients receive instant feedback, making the training experience more intuitive and encouraging them to trust the device's responses.

If the patient 108 fails to press the alert button 112 within a predefined time limit after receiving the visual instruction 118, the medical system 100 is designed to provide additional prompts. In such cases, the medical system 100 can automatically send a follow-up text message reminding the patient 108 to press the alert button 112. This second message might use different wording to capture the patient's attention, such as, “Reminder: Please press the alert button as part of your training. This is your second reminder.” This variation in language helps prevent the messages from being perceived as repetitive or easy to ignore.

In situations where the patient 108 does not respond to multiple training instructions, the medical system 100 can escalate the response by involving a human representative. For instance, if the patient 108 ignores several instructions, a customer support representative could call them directly to understand the issue. During this call, the customer support representative may provide verbal guidance, answer questions, and ensure the patient 108 understands how and when to use the alert button 112. This personalized approach helps address any misunderstandings or technical difficulties the patient 108 might be experiencing, ensuring that they are fully prepared to use the wearable medical device 102 effectively in a real emergency.

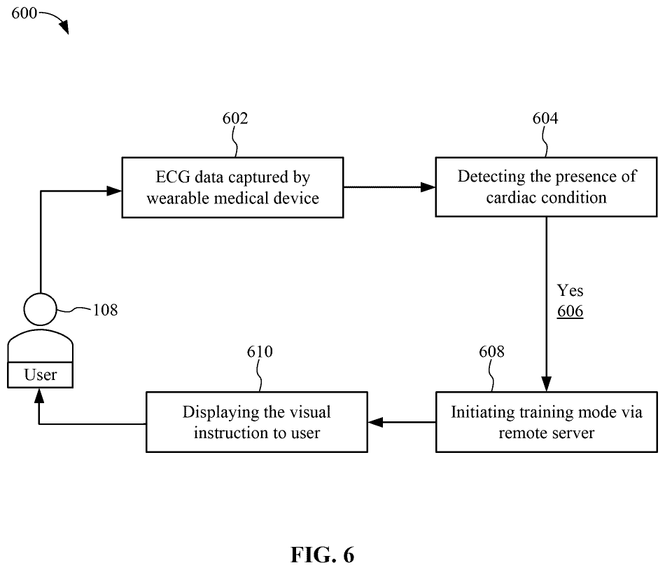

FIG. 6 depicts a block diagram disclosing a process 600 of initiating the training mode based on a predefined training schedule, according to an embodiment of the present disclosure. FIG. 5 is explained in conjunction with previous figures.

The training mode begins with the patient 108 wearing the wearable medical device 102, such as the ASSURE® WCD, which is designed to continuously monitor the patient's heart activity. The wearable medical device 102 is equipped with one or more sensors that capture the patient's ECG data in real-time, at block 602. The ECG data reflects the electrical activity of patient's heart and helps in identifying any abnormal heart rhythms that might indicate a potential cardiac condition. For instance, the wearable medical device 102 can detect if the patient's heart rate becomes dangerously fast or slow.

Once the ECG data is collected, the data is processed by the medical system 100 to analyze the heart's rhythm and check for any signs of cardiac conditions, at block 604. This analysis might involve detecting arrhythmias or other abnormal patterns that could require immediate attention. If a cardiac condition is detected, i.e., affirmative at block 606, the medical system 100 not only prepares to deliver shock therapy if necessary but also initiates a training mode for the patient 108, at block 608. This training is triggered via the remote server 106, such as the Kestra CareStation® remote data platform, which manages the communication between the wearable medical device 102, the patient's mobile device 104, and the remote server 106.

In the training mode, the patient 108 receives the visual instruction 118 on the mobile device 104 and may be displayed on the display 116 of the mobile device 104, at block 610. However, the visual instruction 118 can be delivered in multiple forms to ensure that the patient 108 understands how to respond to the generated alert appropriately. For example, the instructions may be displayed as a text message with simple directives like “Press the Alert Button if you hear this sound!” accompanied by an emoji of the alert button 112. In other cases, a link to an audio file may be included in a text message, allowing the patient 108 to listen to the shock alert sound to familiarize the patient 108 with the alert sound. Alternatively, the visual instruction 118 can be provided in a video format showing a demonstration of pressing the alert button 112 when the alert is generated.

In addition to the above-mentioned messages, the training instruction 118 may also be delivered through a chat interface in the patient application 402 installed on the patient's mobile device 104. For instance, the patient application 402 may send a direct message containing a GIF or an animation that visually demonstrates pressing the alert button 112 to stop the generated alert. This is particularly useful for patient 108 who may be using a mobile device provided by the healthcare service that does not have standard SMS capabilities. The remote server 106, like Kestra CareStation® platform, ensures that these messages are sent timely and understandable, helping the patient 108 to react correctly during an emergency.

Furthermore, the patient application 402 can enhance the patients' experience by providing audio messages that either read out the text instructions or play a recording of the alert sound followed by a directive like “When you hear this sound, press the alert button immediately.” Such multimodal instructions ensure that the patient 108, regardless of their familiarity with technology, can grasp the actions required in an emergency.

This approach to training ensures that the patient 108 is well-prepared to respond to a cardiac emergency by correctly using the alert button 112, thereby improving the chances of timely intervention and preventing unnecessary shocks. For example, if the patient 108 receives the shock alert indicating that an electrical shock is imminent, the patient 108 will know how to press the alert button 112 if he/she is conscious and aware that the alert might be a false positive due to noise or other factors. Such patient-centric design of the medical system 100, combined with real-time training and guidance, significantly enhances the safety and effectiveness of the wearable medical device 102.

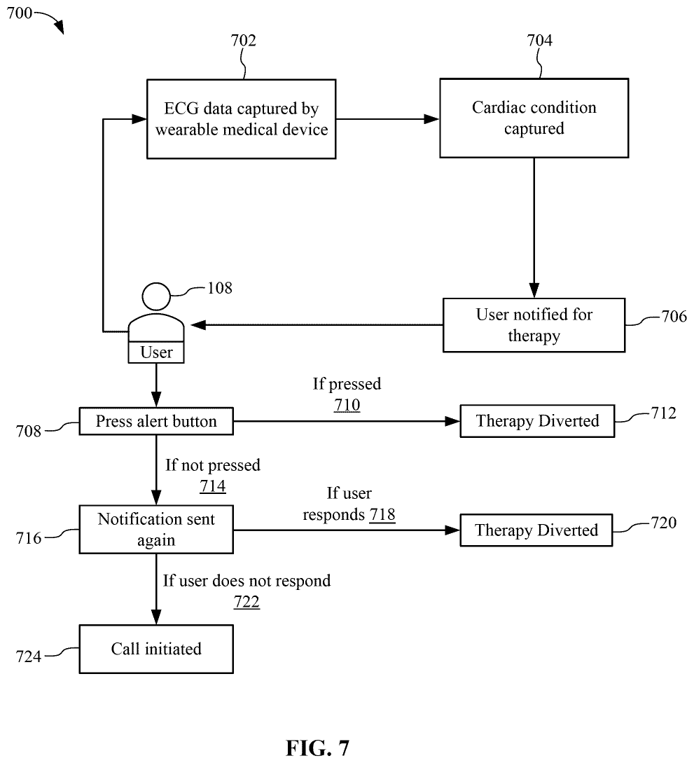

FIG. 7 depicts a block diagram depicting a process 700 when the patient 108 fails to press the alert button 112 during the training mode, according to an embodiment of the present disclosure. Particularly, in FIG. 7, the process 700 exemplifies that a call is initiated if the patient 108 does not respond to a predefined number of visual instructions 118. FIG. 7 is explained in conjunction with previous figures.

The wearable medical device 102, such as the ASSURE® WCD, is designed to continuously monitor the patient's heart activity through embedded sensors. The wearable medical device 102 captures ECG data in real-time (block 702), which reflects the electrical signals of the heart of the patient 108. For instance, if the patient 108 with a history of arrhythmia wears the wearable medical device 102, then variations in the heart rhythm may be detected by the wearable medical device 102, which may indicate a potential cardiac event. The captured ECG data is then processed by the WCD's internal system to identify any signs of dangerous heart conditions (block 704), such as tachycardia (excessively fast heart rate) or bradycardia (excessively slow heart rate).

If the wearable medical device 102 detects a potentially life-threatening cardiac condition, the remote server 106 immediately notifies the patient 108. For example, the wearable medical device 102 might trigger a flashing red light, emit a harsh alarm, and provide a voice message stating, “Preparing to shock. Do not touch the patient.” The notification serves dual purpose, i.e., to alert the patient 108 (block 706) of the imminent therapy and to prepare them for what is about to happen. Further, the notification includes a warning that an electrical shock may be delivered soon if the detected condition is not aborted. In an embodiment, the notification may serve as a means of alerting bystanders or medical personnel in vicinity of the patient 108 for the upcoming therapy delivery. In other embodiments, the patient 108 may inform the bystanders, after being alerted by the notification, of the upcoming therapy delivery.

At this point, the patient 108 is prompted to press the alert button 112 (block 708) if they believe that the alert is a false positive or if they do not feel that their condition is critical enough to require shock therapy. For instance, the patient 108 may have been trained to recognize certain alerts through the visual instruction 118 provided via the patient application 402, which could include videos showing how to press the alert button 112 when they feel fine. The training ensures that the patient 108 knows how to respond quickly and accurately during an emergency.

If the patient 108 presses the alert button 112 (block 710), the mobile device 104 commands the remote server 106 to divert the shock therapy (block 712), preventing the wearable medical device 102 from delivering an unnecessary shock to the patient 108. The medical system 100 confirms this action with a voice message, such as “Shock cancelled,” and a haptic response (e.g., a vibration pattern), via the alert button 112, to reassure the patient 108 that the therapy has been halted or ceased. Additionally, the patient application 402 may display a message like, “Great job! Shock canceled successfully.”

If the patient 108 fails to press the alert button 112 (block 714), a follow-up notification is sent to the patient 108 via the mobile device 104 (block 716). If the patient 108 responds to this follow-up notification (block 718), either by pressing the alert button 112 or by using the patient application 402 to indicate that they feel fine, the shock therapy is again diverted (block 720). The patient application 402 might display a confirmation message, such as “Therapy diverted successfully. Please continue to wear your device.” This approach ensures that false positives do not lead to unnecessary shock delivery and helps the patient 108 feel in control of the situation.

On the other hand, if the patient 108 does not respond (block 722) to either the initial alert or the follow-up notification within a specified time frame, the medical system 100 takes further action by initiating a phone call to the patient's mobile device 104 (block 724). This call might be automated and provide instructions like, “If you are feeling fine, please press the alert button now.” If the patient 108 still does not respond, the medical system 100 may proceed with delivering the shock therapy if the cardiac condition appears critical based on the ECG data of the patient 108.

This method of managing alerts and therapy delivery helps to balance patient 108 safety with the need to minimize unnecessary shocks, enhancing the overall effectiveness of the medical system 100. The combination of visual, audio, and tactile alerts, along with the ability for the patient 108 to cancel therapy, ensures a responsive and patient-friendly experience.

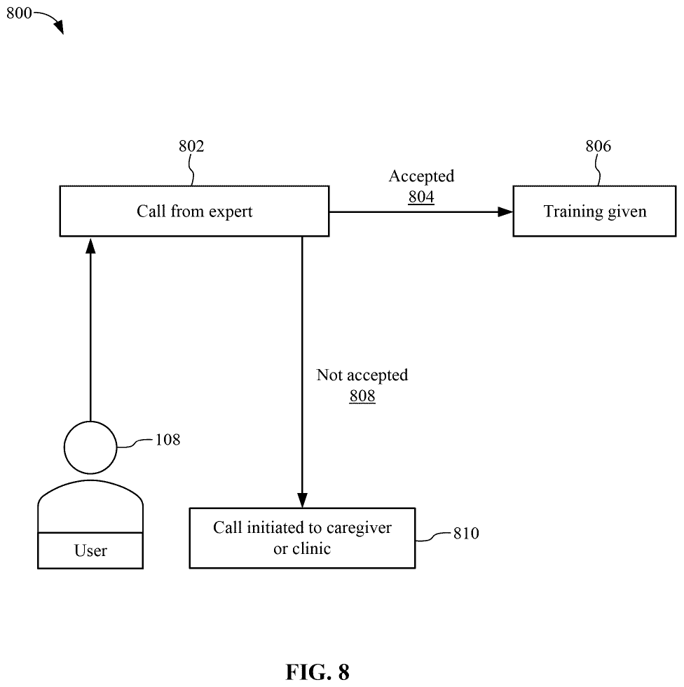

FIG. 8 depicts a block diagram 800 disclosing the initiation of a call to the patient's mobile device 104 when the patient 108 does not respond to the communication received on the mobile device 104, according to an embodiment of the present disclosure. FIG. 8 is explained in conjunction with previous figures.

If the patient 108 does not respond to the notifications sent to the mobile device 104 within a specified time frame, the medical system 100 is designed to automatically initiate a phone call to the patient's mobile device 104. This call acts as a follow-up measure to ensure that the patient 108 is aware of the alert and can take the necessary actions if he/she is conscious and able. For instance, if the patient 108 has not pressed the alert button 112 in response to a training session, the medical system 100 might place a call, at 802, to provide a voice alert, informing the patient 108 about the training session and advising the patient 108 to press the alert button 112 if the patient 108 is feeling fine. This approach ensures that the patient 108 gets a direct and urgent reminder, reducing the risk of missing the alert due to a missed notification.

If the patient 108 accepts this call, at 804, the medical system 100 can provide a quick and guided training session on how to use the alert button 112 effectively, at 806. For example, the call may include a recorded message explaining how to press the alert button 112 if the patient 108 feels alright. Additionally, the call can include questions to assess how the patient 108 is feeling, such as asking if they are experiencing dizziness, chest pain, or any other concerning symptoms. This interactive approach helps in confirming the patient's condition and symptoms and guiding them on the next steps to take. For instance, the message could say, “If you are feeling fine, press the alert button now. If you need help, stay on the line.” This ensures that the patient 108 is both informed and trained.

However, if the patient 108 does not accept the call, at 808, the medical system 100 is configured to escalate the situation by initiating a call to a designated caregiver or a medical expert, at 810. This feature ensures that even if the patient 108 is unable to respond, help can still be dispatched quickly. For example, a caregiver may receive the call, at 810, and the medical system 100 informs them about the current status of the patient 108 and guide to check on them immediately. Alternatively, if the call is directed to a medical expert, the expert may assess the situation based on the real-time ECG data transmitted by the wearable medical device 102 and decide whether to initiate emergency services (e.g., calling 911 for immediate help). This multi-step approach of notifications, direct calls to the patient 108, and escalation to caregivers or experts ensures that the patient 108 gets timely support during critical situations, minimizing the risk of unattended cardiac events.

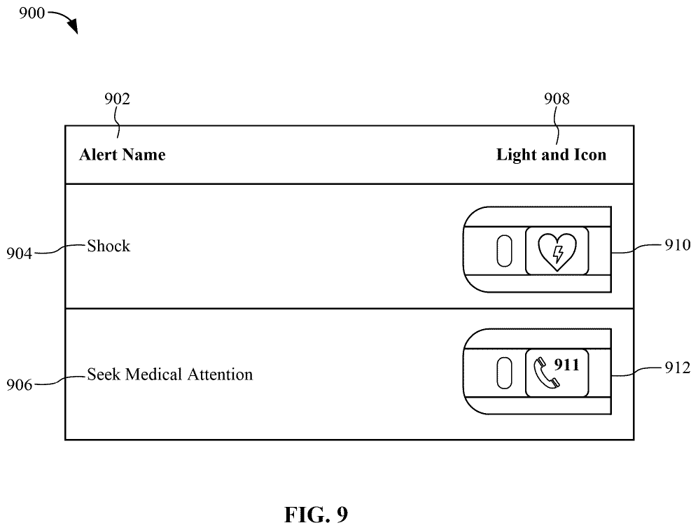

FIG. 9 depicts exemplary buttons 900 provided to the patient 108 to seek medical help during a cardiac event, according to an embodiment of the present disclosure. FIG. 9 is explained in conjunction with previous figures.

The wearable medical device 102 is designed to continuously monitor electrical activity of the patient's heart for cardiac events and also to assess the functionality of the wearable medical device 102 to ensure reliable performance of the wearable medical device 102. When the medical system 100 identifies a cardiac event for the patient 108, in some embodiments, the medical system 100 generates one of two types of alerts, namely, a heart alert or a system alert.

Heart alerts are critical notifications triggered when the medical system 100 detects a heart rhythm that is either too fast or too slow, which could be life-threatening. These alerts require the patient's immediate response. When the heart alert is activated, the patient 108 may see a flashing red light on the wearable medical device 102 or on the display 116 of the mobile device 104. Alternatively, or additionally, the patient 108 may hear a harsh, alternating low-high alarm accompanied by a voice message upon activation of the heart alert. In addition, the patient 108 may experience a predefined number of pulses (e.g., four gentle pulses) followed by an intense vibration (e.g., triple-buzz) from the alert button 112. The purpose of these distinct signals is to ensure that the patient 108 quickly recognizes the urgency of the situation.

System alerts, on the other hand, indicate issues with the equipment itself, such as a misfitting garment worn by the patient 108 or a low battery of the wearable medical device 102. For these alerts, as an example embodiment, the patient 108 may see a blinking yellow light and the alert icon on the monitor, hear a repeating double-tone alarm along with a voice message, and feel a triple-pulse vibration from the alert button 112. While these alerts are less critical than heart alerts, they still require timely attention to ensure that the medical system 100 can continue to function properly.

In FIG. 9, two types of heart alerts 902, namely ‘shock’ alert 904 and ‘seek medical attention’ alert 906 are depicted. Further, light and icon 908 indicates a type of icon displayed corresponding to the respective types of heart alerts 902. When the medical system 100 detects a dangerously fast heart rhythm, the medical system 100 issues the ‘shock’ alert 904 to inform the patient 108 that an electrical shock is about to be delivered. In this scenario, a series of voice messages may play, such as “Preparing to shock. Do not touch the patient” and/or “Preparing to shock in 3, 2, 1.”

If the patient 108 is conscious and notices the ‘shock’ alert 904, the patient 108 must immediately press the alert button 112 to cancel the shock. This action allows the patient 108 to prevent the shock if he/she believes that the shock is not required. When the alert button 112 is pressed, the medical system 100 may confirm the cancellation of the shock with a voice message and/or a vibration. The patient 108 should continue to wear the wearable medical device 102 unless instructed otherwise by a medical professional and should contact emergency services if they feel unwell.

If the patient 108 does not press the alert button 112, the wearable medical device 102 will automatically deliver the shock if considered necessary. The successful delivery of shock is indicated through an icon 910 on the display 116. Following the shock, a sequence of voice messages may play, including “Shock delivered” and “Call 911 now. Do not touch the patient.” The patient 108 may click on an icon 912 to call 911 assistance. In an example embodiment, the medical system 100 may be capable of delivering up to five shocks in a row for a single episode if the dangerous heart rhythm persists. However, the number of shocks permissible for a single episode may be pre-configured.

After the shock is delivered, the medical system 100 continues to analyze the patient's heart rhythm and may issue additional shocks if needed. The medical system 100 also informs any bystanders or caregivers to contact emergency services. Following the shock delivery, voice messages such as “You have received a shock” and “Continue to wear your ASSURE system” may be generated to advise the patient 108 to continue wearing the wearable medical device 102, for continuous monitoring of their heart rate.

In addition, after the shock is delivered, ‘seek medical attention’ alert 906 may be displayed to ensure the patient 108 understands that they received therapy and encourages them to seek medical help immediately. Post-shock, the patient's chest and back may be covered with a conductive gel released by one or more therapy pads to facilitate shock delivery. The gel remains effective for at least an hour, and patients are advised to leave it in place unless a healthcare professional suggests otherwise. Some discomfort or soreness around the chest area is normal after receiving the shock.

If at any time during or after the episode (or cardiac event) the patient 108 hears the ‘shock’ alert 904 again, they should press the alert button 112. The system's voice messages will continue to repeat as needed throughout the episode to guide the patient 108 and ensure they receive appropriate care.

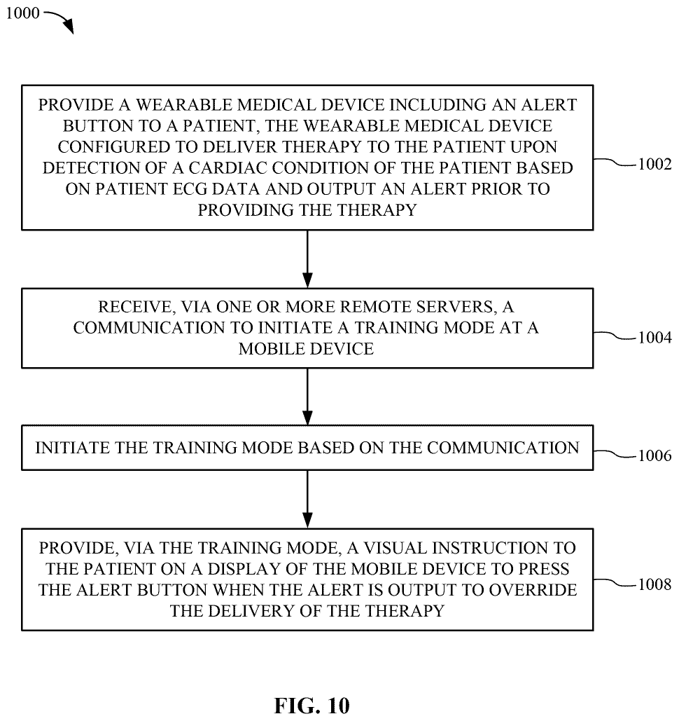

FIG. 10 depicts an example method 1000 to train the patient 108 to override the delivery of shock therapy, according to an embodiment of the present disclosure. The method 1000 is executed by the medical system 100. FIG. 10 is explained in conjunction with previous figures. Although an example utilized for method 1000 depicts a particular sequence of operations, the sequence may be altered without departing from the scope of the present disclosure. For example, some of the operations depicted in FIG. 10 may be performed in parallel or in a different sequence that does not materially affect a function of the method 1000. In other examples, different components of the medical system 100 that executes the method 1000 may perform the operations substantially in parallel or in a specific sequence.

In certain embodiments, as a pre-requisite requirement for transmitting the patient data, a dedicated smartphone or (personal) mobile device 104 is required for relaying the patient data to the remote server 106. As described previously, the mobile device 104 may be configured with the functionality to present visual instruction 118 or the functionality may be provided in an application (e.g., ASSURE® patient application) as part of the medical system 100 (e.g., ASSURE® system) that may be installed/downloaded and run on the patient's personal smartphone or the mobile device 104. However, other applications, medical systems, and wearable medical devices 102 may be utilized without departing from the scope of the present disclosure. The display 116 or the patient application 402 running on the mobile device 104 may include visual elements, audio elements, tactile elements, or a combination thereof for the patient 108 or any person using the patient application 402 or the mobile device 104, to facilitate interaction or communication with the patient or user 108. Further, the patient application 402 may automatically transmit the patient data at certain intervals or when the patient 108 chooses to transmit via the patient application 402. Without limitation, the mobile device 104 may be associated with the patient 108, a family member or someone known to the patient 108, a rescuer, a trained caregiver, or any observer in the vicinity of the patient 108.

The method 1000 begins at block 1002 by providing the wearable medical device 102 including the alert button 112 to the patient 108. The wearable medical device 102 is configured to deliver a shock therapy to the patient 108 upon detection of a cardiac condition of the patient 108 based on patient's ECG data and output an alert before providing the shock therapy.

In certain embodiments, the wearable medical device 102, such as the ASSURE® WCD, may be provided to the patient 108. The wearable medical device 102 is equipped with the alert button 112 and is designed to continuously monitor the patient's heart activity by capturing and analyzing ECG data in real-time. The primary function of the wearable medical device 102 is to detect any abnormal cardiac conditions, such as dangerously fast or slow heart rhythms, which might require immediate intervention. For instance, if the wearable medical device 102 detects a potentially life-threatening arrhythmia, it prepares to deliver the shock to restore a normal heart rhythm. However, before administering the shock, the wearable medical device 102 is configured to generate the alert to notify the patient 108 about the detected condition and the impending therapy.

When the cardiac condition is identified, the wearable medical device 102 triggers a sequence of alerts to ensure that the patient 108 is aware of the situation. The alerts may include a combination of visual, auditory, and tactile signals. For example, a display on the wearable medical device 102 might display a flashing red light accompanied by a harsh, alternating low-high alarm sound, as well as a voice message that warns the patient 108 about the imminent shock. Additionally, the alert button 112 on the wearable medical device 102 may emit a distinctive vibration pattern to capture the patient's attention effectively. This alert system is designed to make sure the patient 108 is fully aware of the critical nature of the situation, even if they are in a noisy environment or have limited visibility.

The alert button 112 provides the patient 108 with an opportunity to intervene. If the patient 108 is conscious and believes that the alert might be a false positive, i.e., their condition is not as critical as the wearable medical device 102 suggests, they can press the alert button 112 to cancel the shock. For instance, if the patient 108 experiences the alerts but feels normal and recognizes it as a potential false alarm, they can quickly press the button 112 to divert the shock therapy. Upon pressing the alert button 112, the wearable medical device 102 confirms the cancellation of the shock through a voice message and a follow-up vibration to reassure to the patient 108 that the shock therapy has been successfully diverted.

This feature not only prevents unnecessary shocks due to false positive detections but also empowers the patient 108 to take control in situations where they might feel well despite the alert being generated. The alert button 112 ensures that the shock therapy is administered only when genuinely needed and allows patients to avoid the discomfort and risks associated with unnecessary shocks. By combining continuous ECG monitoring, a robust alert system, and the alert button's patient intervention capability, the wearable medical device 102 ensures both effective treatment and enhanced patient safety.

The method 1000 further includes receiving, via one or more remote servers 106, a communication to initiate a training mode at the mobile device 104, at block 1004.

The process of initiating the training mode through communication received via the remote server 106 is a crucial feature of the wearable medical device 102 and the medical system 100, designed to enhance patient's preparedness and response capabilities. This process begins when the one or more remote servers 106, such as the Kestra CareStation® remote data platform, send a communication to the patient's mobile device 104, which is paired with the wearable medical device 102. This communication can be triggered by several factors, including a pre-scheduled training frequency, detection of elevated noise levels in the ECG data, or after the patient 108 has experienced a cardiac episode that did not require shock delivery. The goal is to ensure that the patient 108 remains familiar with how to respond to alerts effectively.

The communication from the remote server 106 can manifest in different forms on the mobile device 104. In one embodiment, this communication is delivered as a text message displayed directly on the display 116 of the mobile device 104. For example, the text message may contain straightforward instructions such as, “Press the Alert Button when you hear the alarm,” along with an emoji of the alert button 112 to make the message more recognizable and less intimidating. This simple yet effective approach ensures that even the patient 108 who may not be tech-savvy can quickly understand what action is required.

In another embodiment, the communication is presented as a notification via a dedicated patient application 402 installed on the mobile device 104 of the patient 108. The patient application 402 serves as a centralized platform for managing alerts, viewing ECG data summaries, and receiving training instructions. For instance, the patient application 402 may send a push notification stating, “Training Mode Activated: Please press the Alert Button when prompted,” accompanied by an alert icon to draw the patient's attention immediately. When the patient 108 taps on the notification, the patient application 402 may cause opening a detailed instruction page or a training video demonstrating the steps to follow.

Additionally, the use of the patient application 402 allows for more interactive and multimedia-rich training experiences compared to standard text messages. For example, the patient application 402 might display a GIF or an animation showing a hand pressing the alert button 112 while a voice message plays instructions such as, “This is what you need to do when you hear the alert sound.” In some cases, the patient application 402 may also include a short audio clip of the actual alarm sound that precedes shock delivery, allowing the patient 108 to familiarize themselves with the auditory cue in a controlled, non-emergency setting.

This multi-channel approach to delivering training communications ensures that patients receive the information in a manner that suits their preferences and needs. Whether through a simple text message or a more comprehensive notification via the patient application 402, the medical system 100 is designed to make the training process accessible and effective, thereby increasing the likelihood that patients will respond correctly during real emergency situations.

The method 1000 further includes initiating the training mode based on the communication, at block 1006. Upon receiving the communication, the mobile device 104 activates the training mode automatically. During the training mode, the patient application 402 on the mobile device 104 displays a series of visual instructions 118 to guide the patient 108 through the training process. For example, the patient application 402 might show an instruction 118 that says, “Press the Alert Button when you hear the alarm,” accompanied by a video demonstrating how to press the alert button 112 effectively. This visual instruction 118 can be presented in various formats, such as text messages, MMS, or direct messages (DM) within the patient application 402. In some cases, the communication might include a link to an audio file that mimics the shock alert sound, allowing the patients to familiarize themselves with the auditory cue associated with a real emergency.

To enhance comprehension and engagement, the training mode might also feature animations or GIFs showing a hand pressing the alert button 112, clearly associating this action with stopping the alarm. For example, an animation could depict the alert button 112 being pressed, immediately stopping the alarm sound, and reinforcing the connection between the alert button 112 press and the cessation of the alert. Additionally, audio messages may play instructions like, “When you hear this alert sound, press the Alert Button immediately.” This multimodal approach ensures that the patients with different learning preferences can effectively absorb the training.

If the patient 108 successfully presses the alert button 112 during training, the wearable medical device 102 sends a confirmation signal back to the mobile device 104, which then displays a “Test Successful” message along with positive reinforcement, such as “Great Job! Don't forget to push the Alert Button whenever your ASSURE WCD warns you.” This feedback loop not only confirms that the patient 108 has understood the training but also builds confidence in their ability to manage potential emergencies effectively.

On the other hand, if the patient 108 fails to press the alert button 112 within a predefined time frame, the medical system 100 might resend the training message or escalate the response by initiating a call from a support representative who can provide real-time guidance. This feature ensures that even if the patient 108 misses the initial training prompt, the patient 108 receives adequate support to understand and practice the necessary actions.

The initiation of the training mode based on communication from the remote server 106 represents a strategy to prepare the patient 108 for emergency scenarios, ensuring he/she can respond swiftly and correctly if a real cardiac event occurs.

Finally, the method 1000 includes providing, via the training mode, a visual instruction 118 to the patient 108 on a display 116 of the mobile device 104 to press the alert button 112 when the alert is output, at block 1008.

The training mode of the medical system 100 is designed to ensure that the patient 108 is well-prepared to respond appropriately to alerts, particularly when it comes to using the alert button 112 to override therapy delivery if necessary. When training mode is initiated, the medical system 100 provides clear and detailed visual instructions on the display 116 of the mobile device 104 to guide the patient 108. For example, the patient 108 might receive a push notification from the Kestra CareStation® remote data platform or see a message within the dedicated patient application 402 that reads, “Press the Alert Button when you hear the alert sound.” This message is often accompanied by an instructional video that demonstrates the correct way to press the alert button 112. The video might show a simulation of a hand pressing the alert button 112 while a voiceover explains, “Press and hold the Alert Button if you are feeling okay and want to stop the shock.” Such visual and audio instructions make it easier for patients to understand what they need to do in case of an alert.

The instructional video serves multiple purposes. First, it familiarizes the patient 108 with the alert button's appearance and how to use it effectively. Second, it reinforces the timing of when to press the alert button 112, specifically, when the alert is output before the shock is delivered. By showing a step-by-step demonstration in the video, the training helps reduce panic or hesitation during real emergencies. For instance, the video might illustrate a scenario where the alert button 112 is pressed, causing the alert sound to stop and a confirmation message to appear on the patient application 402, such as, “Alert cancelled. No shock will be delivered.”

Once the patient 108 follows the instructions and presses the alert button 112 during training, the wearable medical device 102 transmits a second communication to the mobile device 104. This communication serves as confirmation that the alert button 112 was pressed correctly. Upon receiving this communication, the mobile device 104 displays a training completion message to the patient 108. For example, the patient application 402 might show a screen with a green checkmark and the text, “Great job! You've successfully completed the training. Remember to press the Alert Button if you feel okay during an alert.” This positive reinforcement not only boosts confidence of the patient 108 but also serves as a reminder of the correct procedure.

The one or more remote servers 106 play a crucial role in managing and scheduling the training sessions. They are capable of automatically initiating training mode based on several factors, ensuring that training is provided when it is most needed. For instance, training might be scheduled at regular intervals, such as weekly or monthly, to keep the patient's response skills sharp. Additionally, the one or more remote servers 106 can trigger training based on the patient's actual use of the wearable medical device 102. If data shows that the patient 108 has not interacted with the alert button 112 recently, a refresher training might be initiated.

Moreover, the one or more remote servers 106 can also schedule training sessions if the ECG data collected by the wearable medical device 102 indicates that a noise threshold has been exceeded. Excessive noise in the ECG signals might suggest that the wearable medical device 102 is at risk of generating a false positive alert. In such cases, a proactive training session is initiated to remind the patient 108 of how to cancel the alert using the alert button 112 if they feel well. For example, a notification might appear saying, “Noise detected in ECG readings. Review how to use the Alert Button to cancel alerts if you feel fine.”

By combining scheduled training, condition-based training, and positive reinforcement upon successful completion, this comprehensive training approach helps ensure that the patients are both confident and competent in managing alerts and potential therapy delivery.

Various embodiments disclosed throughout the present disclosure provide several advantages. Training patients to regulate when the shock therapy should be given creates a balance between safety and comfort. Further, delivering the communication to the respective mobile devices of patients makes it convenient for them to control the functionality of their respective wearable medical devices seamlessly. Furthermore, real time patient data transmission to the remote server(s) enables generation of alert(s) as soon as a cardiac event is detected, making it convenient for the physicians and caregivers to keep a watch on patient's health and about any cardiac episodes.

Other embodiments include combinations and sub-combinations of features described or shown in the drawings herein, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A medical system operable to provide training to a patient, the medical system comprising:
a wearable medical device comprising an alert button, the wearable medical device configured to deliver therapy to the patient upon detection of a cardiac condition of the patient based on patient electrocardiogram (ECG) data and output an alert prior to providing the therapy;
one or more remote servers; and
a mobile device comprising at least one processor and a user interface, the mobile device in communication with the wearable medical device and the one or more remote servers, the at least one processor configured to:
receive a communication to initiate a training mode from the one or more remote servers; and
initiate the training mode based on the communication;
wherein the training mode provides an instruction to the patient through the user interface,
wherein the instruction instructs the patient to press the alert button when the alert is output, and
wherein the alert button is configured to override delivery of the therapy.

2. The medical system of claim 1, wherein the communication comprises a text message provided on the user interface.

3. The medical system of claim 1, wherein the communication comprises a notification provided via a patient application.

4. The medical system of claim 1, wherein the instruction comprises a training video.

5. The medical system of claim 1, wherein the wearable medical device is operable to send a second communication to the mobile device when the patient presses the alert button.

6. The medical system of claim 5, wherein the second communication causes a training completion indication to be provided on the user interface.

7. The medical system of claim 1, wherein the one or more remote servers are operable to schedule initiation of the training mode based on a scheduled training frequency, patient use of the wearable medical device, and/or when the patient ECG data indicates that a noise threshold has been exceeded.

8. The medical system of claim 1, wherein the wearable medical device comprises at least one therapy electrode in communication with an energy source, the at least one therapy electrode configured to deliver a shock to the patient based on the patient ECG data.

9. A method for training a patient to override delivery of a therapy, the method comprising:
providing a wearable medical device comprising an alert button to the patient, the wearable medical device configured to deliver the therapy to the patient upon detection of a cardiac condition of the patient based on patient ECG data and output an alert prior to providing the therapy;
receiving, via one or more remote servers, a communication to initiate a training mode at a mobile device;
initiating the training mode based on the communication; and
providing, via the training mode, an instruction to the patient through a user interface of the mobile device to press the alert button when the alert is output,
wherein the alert button is configured to override the delivery of the therapy.

10. The method of claim 9, wherein the communication comprises a text message provided on the user interface of the mobile device.

11. The method of claim 9, wherein the communication comprises a notification provided via a patient application.

12. The method of claim 9, wherein the instruction comprises a training video.

13. The method of claim 9, further comprising transmitting, via the wearable medical device, a second communication to the mobile device when the patient presses the alert button.

14. The method of claim 13, wherein the second communication causes a training completion indication to be provided on the mobile device.

15. The method of claim 9, wherein the one or more remote servers are operable to schedule initiation of the training mode based on a scheduled training frequency, patient use of the wearable medical device, and/or when the patient ECG data indicates that a noise threshold has been exceeded.

16. A non-transitory computer readable medium, encoded with instructions stored thereon for training a patient to override delivery of a therapy, that when executed by at least one computing device, cause the at least one computing device to perform operations for training the patient to override the delivery of the therapy, the operations comprising;
receiving, via one or more remote servers, a communication at a mobile device;
initiating a training mode for a wearable medical device based on the communication, the wearable medical device comprising an alert button, the wearable medical device configured to deliver the therapy to the patient upon detection of a cardiac condition of the patient based on patient ECG data and output an alert prior to providing the therapy; and
providing, through a user interface of the mobile device, an instruction to the patient to press the alert button when the alert is output,
wherein the alert button is configured to override the delivery of the therapy.

17. The non-transitory computer readable medium of claim 16, wherein the communication comprises a text message provided on the mobile device and/or a notification provided via a patient application.

18. The non-transitory computer readable medium of claim 16, the operations further comprising transmitting, via the wearable medical device, a second communication to the mobile device when the patient presses the alert button.

19. The non-transitory computer readable medium of claim 18, wherein the second communication causes a training completion indication to be provided on the mobile device.

20. The non-transitory computer readable medium of claim 16, wherein the one or more remote servers are operable to schedule initiation of the training mode based on a scheduled training frequency, patient use of the wearable medical device, and/or when the patient ECG data indicates that a noise threshold has been exceeded.
